(19) United States
(12) Patent Application Publication
(10) Pub. No.: US 2018/0353070 A1
(43) Pub. Date: Dec. 13, 2018

(54) Wireless Pads for EKG Heart Monitors

(71) Applicant: Maureen Kratzer, Fort Ogden, FL (US)

(72) Inventor: Maureen Kratzer, Fort Ogden, FL (US)

(21) Appl. No.: 15/617,069

(22) Filed: Jun. 8, 2017

(51) Int. Cl. A61B 5/00; A61B 5/0408

(52) U.S. Cl. CPC A61B 5/0006; A61B 2562/00; A61B 5/6801; A61B 5/0408

(57) ABSTRACT

The current invention is wireless EKG sensors and pads. The pads are sweat proof and hypoallergenic, adhere to patients for up to a week, and are labeled to direct the user where to place each pad. The EKG sensors wirelessly transmit the test data. This eliminates test disruptions from motion-related static, while allowing 24/7 EKG monitoring and opening a new realm of heart arrhythmia testing. Further, by being wireless and sweat proof, EKG monitoring can be more easily conducted during strenuous activity.

### CROSS-REFERENCES TO RELATED APPLICATIONS (IF ANY)

None.

### BACKGROUND

### 1. Field of the Invention

This invention relates to health monitoring pads and particularly monitoring pads which communicate data wirelessly.

### 2. Description of Prior Art

An electrocardiogram (EKG) measures electrical impulses generated by heartbeats. This provides information such as heart rate, rhythm, and information about the heart chambers. An EKG test collects data using sensor pads placed at 10 specific locations on the body.

Each of the sensor pads has a wire that connects the sensor to the EKG device. The presence of wires creates tangles, which can delay work progress for medical staff. The wires also restrict a patient's movement, as static charges from movement can disrupt transfer of data. Due to this restriction, the wires are placed before each test and removed directly after. In hospital settings, this creates excess work and potential problems when dealing with infectious patients. Infectious patients require isolation, and the entire device much be sanitized after each use.

There is still room for improvement in the art.

### SUMMARY OF THE INVENTION

The current invention is wireless EKG pads. The pads communicate the EKG test data wirelessly to reduce the need to place and remove sensors on patients.

The EKG pads consist of an electrode sensor and a gel pad. The gel pad would adhere to the patient's skin, but can be easily removed. The pads are sweat proof and hypoallergenic, to allow use while engaging in strenuous activities.

Each pad has an electrode sensor to collect the electrical measurements related to the cardio system. Each pad would have a wireless transmitter to send readings wirelessly to the EKG device. Wireless sensors will reduce the loss of data due to static charges.

Each pad is labeled for which body position it is to be placed.

### BRIEF DESCRIPTION OF THE DRAWINGS

Without restricting the full scope of this invention, the preferred form of this invention is illustrated in the following drawings:

FIG. 1 shows the bottom of the EKG sensor pads;

FIG. 2 the top of the EKG sensor pads;

FIG. 3 shows the EKG sensor pads;

FIG. 5 shows a side bottom view of the EKG pad;

FIG. 6 shows another view of the EKG sensor pad;

FIG. 7 shows the EKG sensor communicating with an EKG device; and

FIG. 8 shows the EKP sensor next to a quarter.

### DESCRIPTION OF THE PREFERRED EMBODIMENT

There are a number of significant design features and improvements incorporated within the invention.

The current invention is wireless EKG pads. The pads communicate the EKG test data wirelessly to reduce the need to place and remove sensors on patients.

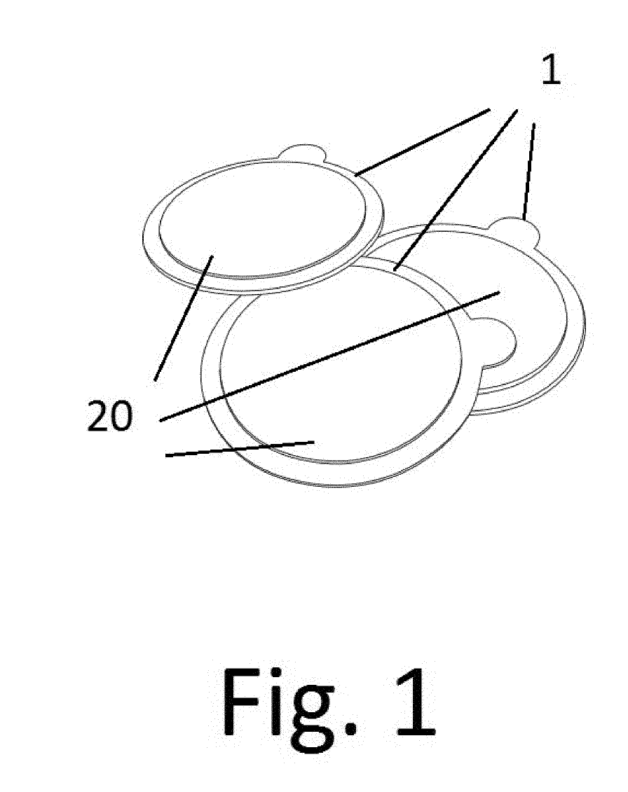

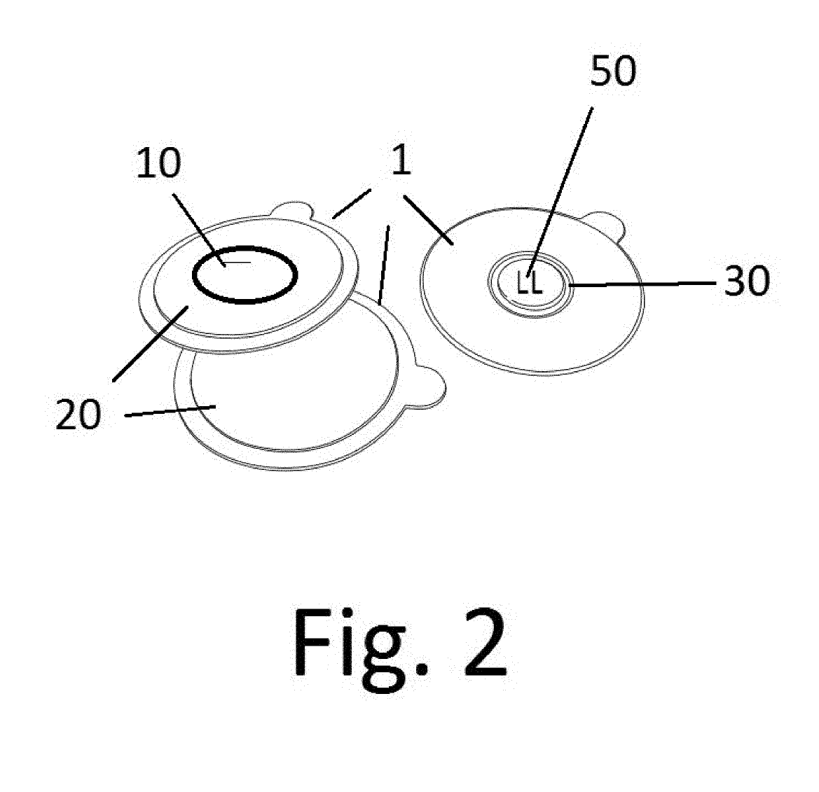

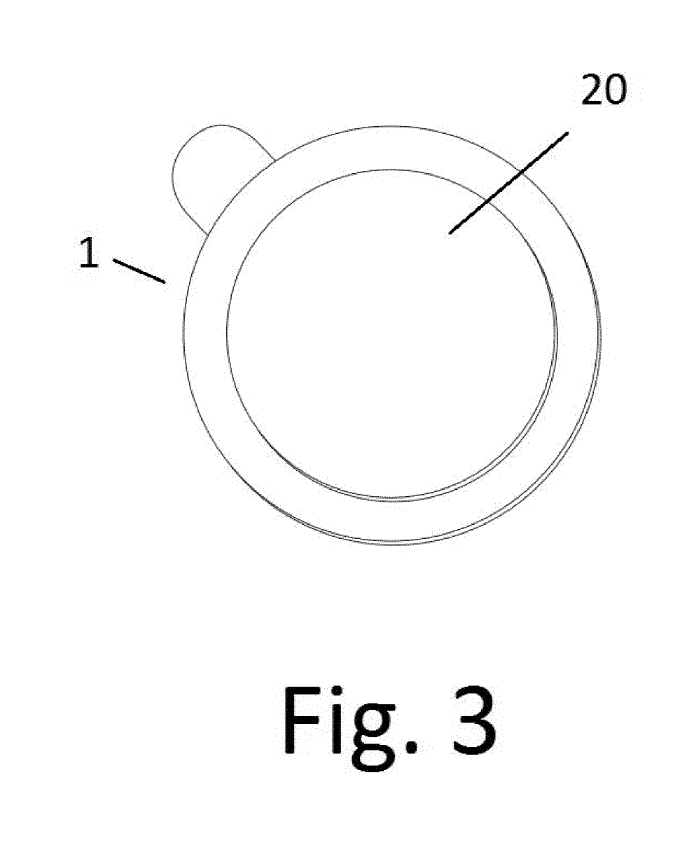

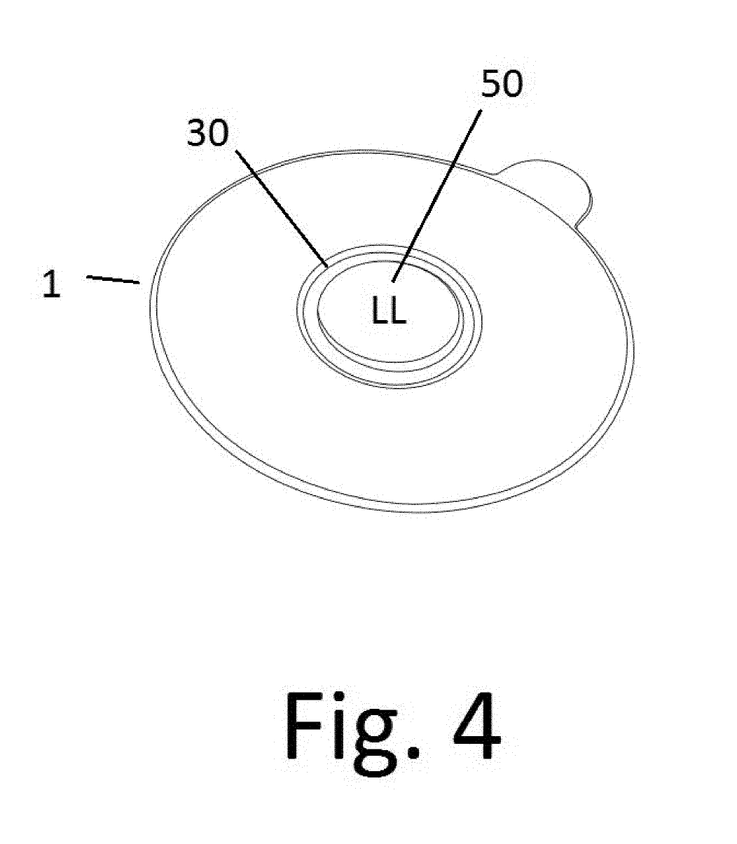

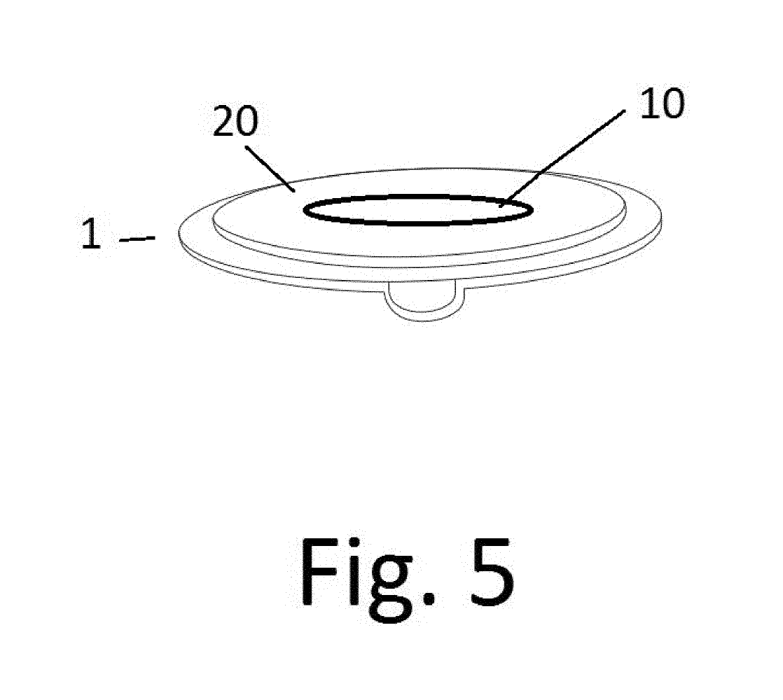

As shown in FIGS. 1 through 6, the EKG pads 1 consist of an electrode sensor 10 and a gel pad 20 on the bottom side of the pads 1. The gel pad 20 would adhere to the patient's skin, but can be easily removed. The pads 1 are sweat proof, to allow use while engaging in strenuous activities. In the preferred embodiment, the gel used in the gel pad 20 would be hypoallergenic.

The EKG pads 1 are flat and circular in shape with a top side and a bottom side.

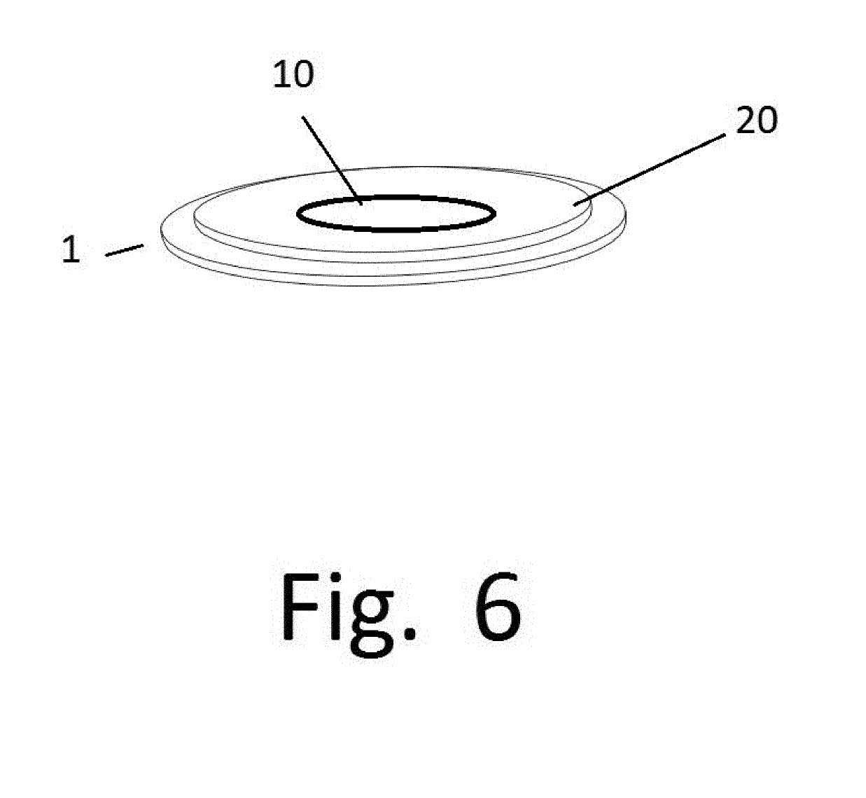

Each pad 1 has an electrode sensor 10 to collect the electrical measurements related to the cardio system. Each pad 1 would have a wireless transmitter 30 connected to the sensor 10 to send readings to the EKG device 300 as shown in FIG. 7. The wireless transmitter 30 sends the transcribed data from the readings to the EKG device 300 wirelessly. Wireless sensors will reduce the loss of data due to static charges.

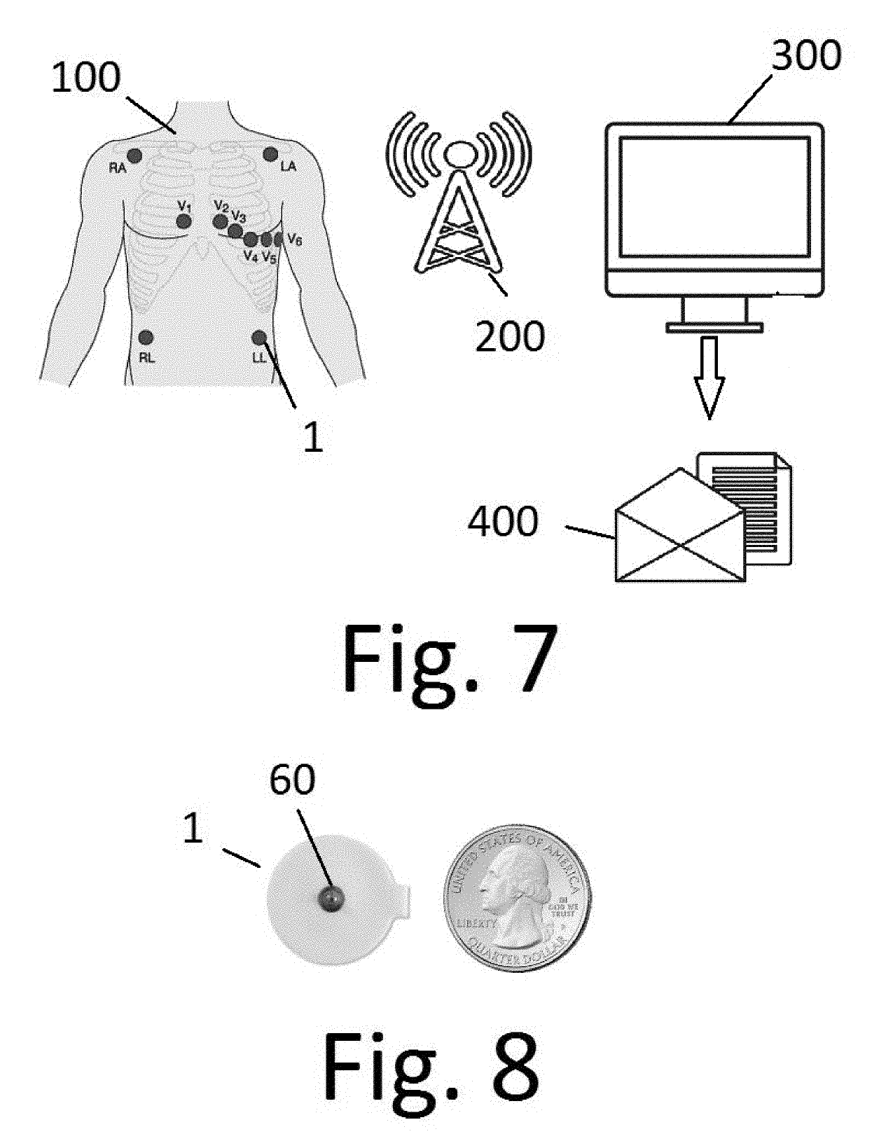

Each pad 1 may be labeled 50 for which body position it is to be placed on a person's body 100 as shown in FIG. 7. It will use wireless communication 200 to the EKG device 300. The pad 1 wirelessly sends transcribed reports with an icon showing where the electrodes where placed. These medical reports can be sent to and reviewed by doctors or the medical staff. The transcribed reports can be sent to an E-mail 400 or SMS address. This eliminating guess work and wait time for answers. The pad 1 would be slightly smaller than the size of a quarter as shown in FIG. 8.

The pad 1 has a power source 60 that powers the EKG pad 1, the sensor 10 and its wireless transmitter 30. In the preferred embodiment is a battery.

### Advantages

The innovative EKG pads reduce health worker labor, reduce room for error, reduce data lost to static charges, reduce opportunity for infections, and increase the range of activities that EKG tests can be reliably used during. Current EKG sensors have wire leads, which often get tangled and require removal after each test. During a test, any movements can lead to static discharge, which may disrupt test data. When the sensors and leads are moved from one area to another, infections can be spread. By having wireless EKG sensors, this is eliminated. As they are wireless, the sensors may remain on a patient for up to a week, and it is unlikely that movement would create static discharge, thus reducing disrupted data. Risk of transferring infectious diseases is eliminated, as there are no devices or wires that will be shared between patients.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided. With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

That which is claimed is:

1. A device comprising:
An EKG pad with a top side and a bottom side with a gel pad on the bottom of said EKG pad with a sensor connected to a wireless transmitter.

2. The device according to claim 1 further comprising:
Where the sensor is an electrode sensor to collect electrical measurements related to a cardio system.

3. The device according to claim 1 further comprising:
Having a label on the top of said EKG pad.

4. The device according to claim 1 further comprising:
Having the wireless transmitter transmit data wirelessly to an EKG device.

5. The device according to claim 1 further comprising:
Having the EKG pads attached to a patient's body.

6. The device according to claim 5 further comprising:
Where the EKG pads are attached at specific points on the body.

7. The device according to claim 6 further comprising:
Where the EKG pads are labeled with the location the pad is to be placed.

8. The device according to claim 1 further comprising:
Having a power source.

9. The device according to claim 8 further comprising:
Where the power source is a battery.

10. A device comprising:
An EKG pad with a top side and a bottom side with a gel pad on the bottom of said EKG pad with a sensor connected to a wireless transmitter where the sensor is an electrode sensor to collect electrical measurements related to a cardio system where the wireless transmitter transmit data wirelessly to an EKG device and having the EKG pads attached to a patient's body.

11. The device according to claim 10 further comprising:
Having a label on the top of said EKG pad.

12. The device according to claim 10 further comprising:
Where the EKG pads are attached at specific points on the body.

13. The device according to claim 10 further comprising:
Where the EKG pads are labeled with the location the pad is to be placed.

14. The device according to claim 10 further comprising:
Having a power source.

15. The device according to claim 14 further comprising:
Where the power source is a battery.

16. The device according to claim 10 further comprising:
Where the EKG pad is circular in shape.

17. The device according to claim 10 further comprising:
Where the gel in the gel pad is hypoallergenic.

18. The device according to claim 10 further comprising:
Where the data is sent to an E-mail address.

18. The device according to claim 10 further comprising:
Where the data is sent in a SMS message.
